(19) United States
(12) Patent Application Publication
(10) Pub. No.: US 2008/0132740 A1
(43) Pub. Date: Jun. 5, 2008

(54) Solvent For Cleaning Semiconductor Manufacturing Apparatus

(75) Inventors: Tomoyuki Hirano, Kanagawa (JP); Masaaki Yoshida, Kanagawa (JP)

Correspondence Address: THELEN REID BROWN RAYSMAN & STEINER LLP, P. O. BOX 640640, SAN JOSE, CA, 95164-0640, US

(73) Assignee: Tokyo Ohka Kogyo Co., Ltd., Kanagawa (JP)

(21) Appl. No.: 11/792,467

(22) PCT Filed: Nov. 8, 2005
(86) PCT No.: PCT/JP05/20446

§ 371 Date: Jun. 6, 2007

(30) Foreign Application Priority Data
Dec. 6, 2004 (JP) 2004-353375

(51) Int. Cl. C07C 31/125; C07C 31/12

(52) U.S. Cl. 568/840

(57) ABSTRACT

A cleaning solvent is disclosed for removing residual resin compositions. The cleaning solvent contains at least an alcohol solvent having a boiling point of at least 100° C. The alcohol solvent having a boiling point of at least 100° C. is preferably constituted of at least one solvent selected from n-butyl alcohol, isobutyl alcohol, n-pentanol, 4-methyl-2-pentanol, and 2-octanol. It is more preferable that the alcohol solvent is an isobutyl alcohol.

### TECHNICAL FIELD

The present invention relates to cleaning solvents, specifically to cleaning solvents for semiconductor production apparatuses, and in particular to cleaning solvents used for cleaning the charging lines of semiconductor production apparatuses at the stage in which the topcoat protective films for application in liquid immersion lithography processes are formed.

### BACKGROUND ART

Lithography methods have been frequently used for the production of fine features in various kinds of electronic devices, such as semiconductor devices and liquid crystal devices. However, as the device features are further miniaturized, having miniaturized resist patterns in lithography processes will also desirable.

In the advanced field, for example, a lithography process now allows the formation of a fine resist pattern having a line width of about 90 nm. However, finer pattern formation will be required in the future.

For attaining the formation of such a fine pattern having a line width of less than 90 nm, a first step is to develop a lithography device and a corresponding resist. Common factors to consider for developing the lithography device include shortening of the wavelengths of the light source such as an F2 laser, EUV (extreme UV light), electron beam, and X-ray, and increasing the numerical aperture (NA) of the lens.

However, the shortening of the optical wavelength may require a new and more expensive lithography device. In addition, due to an inverse relationship between the resolution and the focal depth width, even if the resolution is increased, a disadvantage occurs at high NA in which focal depth width decreases. Recently, a method known as a liquid immersion lithography process has been reported (e.g., Non-Patent Documents 1, 2, and 3) as a lithography technology to solve such problems. In this process, a liquid such as purified water or a fluorine-based inert liquid (immersion liquid) is placed on a resist film in a predetermined thickness between a lens and the resist film. In this method, the space of the path of exposure light, which is conventionally filled with inert gas such as air or nitrogen, is replaced with a liquid having a higher refractive index (n), for example purified water, to attain high resolution without a decrease in focal depth width, similar to the use of a light source of shorter wavelength or a high NA lens, even if an optical source having the same exposure wavelength is employed.

Such liquid immersion lithography has been given considerable attention because its use allows a lens implemented in the existing device to realize the formation of a resist pattern superior in higher resolution property as well as excellent in focal depth in low costs.

However, in the abovementioned liquid immersion lithography process in which exposure is conducted under conditions where a medium such as purified water is interposed between a lens and a substrate, although the materials used in a conventional lithography process may be utilized without any adjustment, it is suggested to use materials different from those of conventional lithography process. In particular, a process in which an alkali-soluble polymer is used for a topcoat resist protective film is taken notice. However, forming such a topcoat protective film requires cleaning of the charging lines of semiconductor manufacturing apparatuses, every time materials for the topcoat protective film are provided. As cleaning solvents for semiconductor manufacturing apparatuses, isopropyl alcohol and ketone solvents have been mainly used (e.g., Patent Documents 1, 2, and 3). However, when such conventional cleaning solvents are used, problems occur regarding cleaning capability, defective coating in a resin coating step in which the resin is passed through the charging lines after cleaning, insufficient transparency of the topcoat protective film after coating, insufficient purity of the cleaning solvents themselves, and difficulty reducing the amount of solvents used.
(Non Patent Document 1) Journal of Vacuum Science & Technology B (J. Vac. Sci. Technol. B) (Issued country: U.S.A.), Vol. 17, No. 6, pages 3306-3309, 1999.
(Non Patent Document 2) Journal of Vacuum Science & Technology B (J. Vac. Sci. Technol. B) (Issued country: U.S.A.), Vol. 19, No. 6, pages 2353-2356, 2001.
(Non Patent Document 3) Proceedings of SPIE (Issued country: U.S.A.), Vol. 4691, pages 459-465, 2002.
(Patent Document 1) Japanese Unexamined Patent Application Publication No. H06-069175
(Patent Document 2) Japanese Unexamined Patent Application Publication No. H07-003294
(Patent Document 3) Japanese Unexamined Patent Application Publication No. H11-174691

### DISCLOSURE OF THE INVENTION

### Problems to be Solved by the Invention

The present invention has been made in view of the above, and aims to provide cleaning solvents, in particular cleaning solvents used for cleaning charging lines of semiconductor production apparatuses at the stage in which topcoat protective films for application in liquid immersion lithography processes are formed.

### Means for Solving the Problems

The present inventors have thoroughly investigated cleaning solvents to solve the above described problems. As a result, the present inventors have found that a certain solvent having a boiling point of at least 100° C. is suited to clean semiconductor production apparatuses at the stage in which topcoat protective films for application in liquid immersion lithography processes are formed, and the present invention has been completed on the basis of this finding.

Thus, the cleaning solvent of the present invention is a cleaning solvent for resin compositions, used for cleaning and removing residual resin compositions from sites on which multiple types of resin compositions contact sequentially, the solvent comprising at least an alcoholic solvent having a boiling point of at least 100° C.

### EFFECTS OF THE INVENTION

The present invention may provide a cleaning solvent for resin compositions having a superior cleaning capability and allows protective films to be coated appropriately after the cleaning. In addition, the amount of the cleaning solvent used may be reduced, thereby also reducing particles in the cleaning solvent for resin compositions, therefore allowing for topcoat protective films to be formed with high transparency. Consequently, high-resolution resist patterns can be obtained by way of forming the topcoat protective films applied in liquid immersion lithography processes.

### PREFERRED MODE FOR CARRYING OUT THE INVENTION

The cleaning solvent according to the present invention may solve problems in liquid immersion lithography, such as the cleaning of semiconductor production apparatuses having imperfections, and different materials requiring an excess amount of dummy dispense, in that it contains at least an alcohol solvent having a boiling point of at least 100° C., which allows high-resolution resist patterns, by way of liquid immersion lithography, to be formed.

In view of the cleaning capability, the alcoholic solvent in the cleaning solvent of the present invention is preferably at least one selected from isobutyl alcohol, n-butyl alcohol, 3-methyl-2-butyl alcohol, 2-methyl-1-butyl alcohol, 2-ethyl-1-butyl alcohol, 3-pentanol, 4-methyl-2-pentanol, 2-ethylhexanol, n-hexanol, cyclohexanol, 2-methylcyclohexanol, 2-heptanol, 3-heptanol, n-heptanol, 3,5-dimethyl-1-hexine-3-ol, n-octanol, 2-octanol, n-amyl alcohol, sec-amyl alcohol, tert-amyl alcohol, isoamyl alcohol, glicydol, n-decanol, tetrahydrofurfuryl alcohol, furfuryl alcohol, α-terpineol, neo-pentyl alcohol, 1-nonanol, and 3-methyl-1-butyne-3-ol, is more preferably at least one selected from n-butyl alcohol, isobutyl alcohol, n-pentanol, 4-methyl-2-pentanol, and 2-octanol, and is most preferably isobutyl alcohol.

The cleaning solvent of the present invention is preferably manufactured by filtrating with a filter material. The filtration process by way of the filter material may provide a cleaning solvent that is itself of high purity and far from being a source of pollution, assure the transparency of the topcoat protective film, and decrease the amount of the cleaning solvent required.

In view of contaminant removal capability, the pore size of the filter material is preferably 0.01 μm to 0.10 μm, and is more preferably 0.02 μm to 0.05 μm.

The filter material may be selected from various filter materials without limitation, as long as it is capable of forming a filter having the pore size described above. Examples of the filter materials include polyethylene, polytetrafluoroethylene, polypropylene, nylon, and the like.

The cleaning solvent of the present invention is characterized in that one of the resin compositions constitutes a topcoat protective film of a resist film. The topcoat protective film is preferably for application in liquid immersion lithography processes.

Such a topcoat protective film is preferably formed from materials comprising an alkali-soluble polymer component. The polymer components include a polymer having a constitutional unit of cyclic fluoroalcohol represented by the general formula (1):

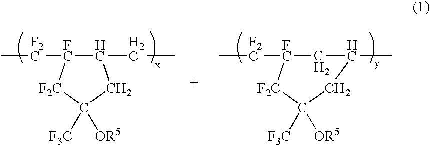

Other polymeric components may be an acrylic polymer. The acrylic polymer preferably has at least a constitutional unit represented by the general formula (2):

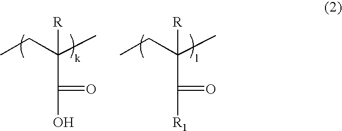

in which R is selected from a hydrogen atom, a methyl group, and hydroxyalkyl group having 1 to 5 carbon atoms, R₁ is hydrocarbon group having at least one aliphatic structure, k and l represent the mole % of each constitutional unit, and are 5 to 95 mole %, respectively.

R₁ in the general formula (2) is preferably at least a hydrocarbon group selected from a cyclohexyl group, an adamantyl group, a norbornyl group, an isobornyl group, a tricyclodecyl group, and a tetracyclododecyl group.

R₁ is preferably selected from tricyclodecyl and cyclohexyl.

The acrylic polymer preferably has at least a constitutional unit in which a third constitutional unit is added to the constitutional unit represented by the general formula (2), and is represented by the general formula (3):

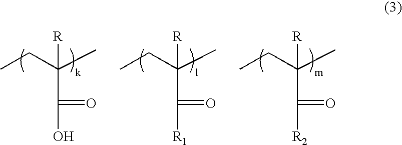

in which R is selected from a hydrogen atom, a methyl group, and hydroxyalkyl group having 1 to 5 carbons atoms, R₁ is hydrocarbon group having at least one aliphatic structure, k, l, and m represent the mole % of each constitutional unit, and are 5 to 50 mole %, respectively.

R₂ in the general formula (3) is preferably at least one selected from an alkyl group and a hydroxyl alkyl group having a carbon number of 1 to 5. R₂ is preferably selected from n-butyl and isobutyl group.

k is 5 to 90 mole %, l is 5 to 90 mole %, and m is 5 to 90 mole %.

The remaining resin composition is a resist composition which forms a resist film. Such resist composition may be a conventional positive or negative type resist composition.

### EXAMPLES

The present invention is explained in detail with reference to the below Examples. The present invention should not be construed in any way as to be limited to the below Examples.

Using isobutyl alcohol (IBA) as the cleaning solvent of the present invention, the reduction of particles from the cleaning solvent through filtration by various filter materials made of polyethylene (PE), polypropylene (PP) and nylon was investigated.

### Examples 1 to 4

The filter materials having the various pore sizes (in parenthesis) shown in Table 1 below were used as the various filter materials. More specifically, by filtering IBA through filters made of PE (0.05 μm, Example 1), PP (0.05 μm, Example 2), nylon (0.04 μm, Example 3), and PP (0.02 μm, Example 4), respectively, cleaning solvents were obtained.

The particles in these cleaning solvents were measured using a particle counter. The results are shown in Table 1.

TABLE 1
Measurement Result of Particle Counter

| | | | Pore | | | | | |
|---|---|---|---|---|---|---|---|---|
| | Sol- | Filter | Size | Size of Particles (μm) | | | | |
| | vent | Material | (μm) | ≧0.135 | ≧0.15 | ≧0.2 | ≧0.22 | ≧0.3 |
| Ex. 1 | IBA | PE | 0.05 | 11.4 | 8.1 | 3.0 | 1.9 | 0.6 |
| Ex. 2 | IBA | PP | 0.05 | 7.7 | 3.3 | 1.2 | 0.8 | 0.1 |
| Ex. 3 | IBA | Nylon | 0.04 | 9.2 | 6.7 | 2.9 | 2.2 | 1.0 |
| Ex. 4 | IBA | PP | 0.02 | 5.2 | 2.8 | 1.0 | 0.8 | 0.4 |

unit: particle number/ml

The results of Table 1 demonstrate that particles contained in IBA are greatly reduced by use of the various filter materials described above.

### Example 5

Formation of a topcoat protective film was investigated in relation to a resist film to which the cleaning solvent according to the present invention had been applied.

The resin component, the acid generator, and the nitrogen-containing organic compound described below, were dissolved homogeneously into an organic solvent to prepare a resist composition.

The copolymer consisting of the constitutional units, expressed by the chemical formula (4) shown below, was used in an amount of 100 parts by mass as the resin component. The ratio of constitutional units l, m, and n for preparing the resin component was that l is 20 mole %, m is 40 mole %, and n is 40 mole %.

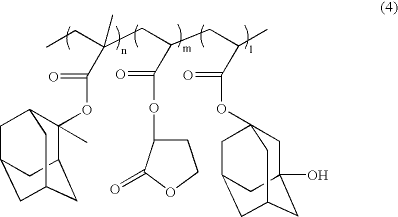

2.0 parts by mass of triphenylsulfonium nonafluorobutanesulfonate and 0.8 parts by mass of tri(tert-butylphenyl)sulfonium trifluoromethane sulfonate were used as the acid generator.

In addition, an aqueous solution with a 7.0% concentration of a mixed solvent of propyleneglycol monomethylether and propyleneglycol monomethylether acetate (mixture ratio=6:4) was used as the organic solvent. Moreover, 0.25 parts by mass of triethanolamine were used for the nitrogen-containing organic compound. Furthermore, 25 parts by mass of gamma-butyrolactone were mixed as an additive.

A resist pattern was formed using the resist composition prepared as described above. Initially, an organic composition of antireflection coating ARC29 (commercial name, by Brewer Co.) was coated onto a silicon wafer using a spinner, followed by heating at 205° C. on a hot plate for 60 seconds for drying, thereby forming an organic antireflection film having a film thickness of 77 nm. Then, the resist composition described above was coated on the antireflective film using a spinner, followed by pre-baking at 130° C. on a hot plate for 90 seconds for drying, thereby forming a resist film having a film thickness of 225 nm on the antireflective film.

100 parts by mass of the copolymer of which the constitutional unit is a cyclic fluoroalcohol expressed by the chemical formula (1) (molecular weight: 13,800, all R⁵s are hydrogen atoms, x:y=50:50 mole %), 5 parts by mass of a cross-linker (tetrabutoxy methylated glycoluril), and 0.6 parts by mass of (CF₂)₃(SO₂)₂NH were dissolved into 2-methyl-1-propyl alcohol to prepare a material for a protective film having a resin concentration of 2.8%.

A charging line of a semiconductor production apparatus was cleaned with the IBA obtained in Example 1. The material for a protective film was rotary-coated on the resist film by passing through the cleaned charging line, followed by heating at 90° C. for 60 seconds to form a protective film having a film thickness of 70.0 nm.

Next, a pattern light was irradiated (exposed) through a mask pattern using an ArF excimer laser (wavelength 193 nm) of a Nikon-S302A lithography machine (by Nikon Co.). Then, as the treatment of liquid immersion lithography, purified water was dripped continuously onto the resist film at 23° C. for 2 minutes while rotating the silicon wafer on which the exposed resist film had been disposed. This step corresponds to a step of an actual production process, where the exposure is carried out in a completely immersed state. However, it is also theoretically possible that the exposure itself can be completely carried out in optical systems on the basis of analysis in terms of the prior liquid immersion lithography. Therefore, this step was conducted simply in a way in which the resist film is first exposed, and then purified water that is a refractive index liquid (immersion liquid) was added onto the resist film after the exposure, so as to evaluate only the influence of the immersion liquid to the resist film.

After the purified water dripping step, PEB treatment was carried out under conditions of 115° C. for 90 seconds. After the PEB treatment, the film was allowed to stand in an environment without an amine filter for 15 minutes, and was then placed in an exposure room for 20 minutes. These processing conditions of standing correspond to a state of standing for 20 minutes under a normal atmosphere (2 to 4 ppm of amine concentration). After the process of standing, development was carried out using an alkaline developer at 23° C. for 60 seconds while keeping the protective film still. The alkaline developer was an aqueous solution of 2.38% by mass tetramethylammonium hydroxide. The developing step could remove the protective film completely, and the resist film could be developed properly.

The resulting 130 nm resist pattern of 1:1 line-and-space was observed under a scanning electron microscope (SEM), which revealed that the pattern profile was the proper rectangular shape.

### Comparative Example 1

A pattern was formed in the same way as Example 6, except that the IBA used for cleaning the charging line in Example 1 was replaced with isopropyl alcohol (IPA).

The resulting 130 nm resist pattern of 1:1 line-and-space was observed under a scanning electron microscope (SEM), which revealed that the proper rectangular shape could not be obtained. It is believed that the pattern profile had nonuniform film thickness due to the influence of cleaning capability of the cleaning solvent.

### INDUSTRIAL APPLICABILITY

As described above, the cleaning solvents according to the present invention can exhibit superior cleaning capability and lead to the proper coating of topcoat protective films after cleaning. Moreover, the particles are greatly reduced in the cleaning solvents according to the present invention, and therefore may be appropriately applied to form the topcoat protective films of resist films used for immersion lithography processes or resist films of dry thin films, etc., thereby to producing high-resolution resist patterns.

1. A cleaning solvent for resin compositions, used for cleaning and removing remaining resin compositions from sites with which multiple kinds of resin compositions sequentially contact, the solvent comprising at least an alcoholic solvent having a boiling point of at least 100° C.

2. The cleaning solvent according to claim 1, wherein the alcoholic solvent is at least one selected from n-butyl alcohol, isobutyl alcohol, n-pentanol, 4-methyl-2-pentanol, and 2-octanol.

3. The cleaning solvent according to claim 1, wherein the alcoholic solvent is isobutyl alcohol.

4. The cleaning solvent according to claim 1, wherein the alcoholic solvent is manufactured by filtrating by way of a filter material.

5. The cleaning solvent according to claim 4, wherein the pore size of the filter material is 0.01 μm to 0.10 μm.

6. The cleaning solvent according to claim 4, wherein the filter material is at least one selected from polyethylene, polytetrafluoroethylene, polypropylene, and nylon.

7. The cleaning solvent according to claim 1, wherein one of the multiple kinds of resin compositions constitutes a topcoat protective film of a resist film.

8. The cleaning solvent according to claim 7, wherein the topcoat protective film is for applying to a liquid immersion lithography process.

9. The cleaning solvent according to claim 7, wherein the topcoat protective film comprises an alkali-soluble polymer component.

10. The cleaning solvent according to claim 9, wherein the alkali-soluble polymer component is a polymer having a constitutional unit of cyclic fluoroalcohol represented by the general formula (1):

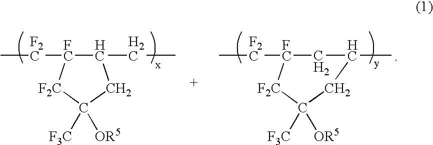

11. The cleaning solvent according to claim 9, wherein the alkali-soluble polymer component is an acrylic polymer.

12. The cleaning solvent according to claim 11, wherein the acrylic polymer has at least a constitutional unit represented by the general formula (2): 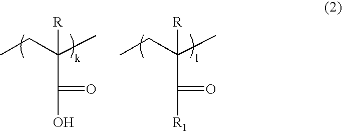
wherein R is selected from a hydrogen atom, a methyl group, and a hydroxyalkyl group having 1 to 5 carbon atoms; R₁ is a hydrocarbon group having at least one aliphatic structure; and k and l represent the mole % of each constitutional unit, each being 5 to 95 mole %.

13. The cleaning solvent according to claim 12, wherein a third constitutional unit is added to the constitutional unit represented by the general formula (2), and is represented by the general formula (3): 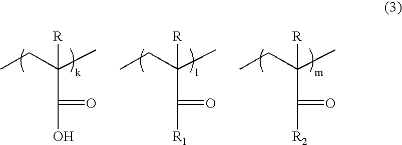
wherein R is selected from a hydrogen atom, a methyl group, and a hydroxyalkyl group having 1 to 5 carbon atoms; R₁ is a hydrocarbon group having at least one aliphatic structure; k, l, and m represent the mole % of each constitutional unit, each being 5 to 50 mole %.
